(19) United States
(12) Patent Application Publication
(10) Pub. No.: US 2022/0296265 A1
(43) Pub. Date: Sep. 22, 2022

(54) Skin Tag removal System and Kit

(71) Applicant: Veronica Rutt, Nazareth, PA (US)

(72) Inventor: Veronica Rutt, Nazareth, PA (US)

(21) Appl. No.: 17/670,229

(22) Filed: Feb. 11, 2022

Related U.S. Application Data
(60) Provisional application No. 63/163,625, filed on Mar. 19, 2021.

(51) Int. Cl. A61B 17/3201; A61B 17/29; A61K 31/167; A61L 24/02; A61F 13/40

(52) U.S. Cl. CPC A61B 17/3201; A61B 17/29; A61K 31/167; A61L 24/02; A61M 35/006; A61B 2017/00761

(57) ABSTRACT

A system for a safe and effective removal of skin tags at home includes a kit that contains the necessary instruments and medications for the safe and effective self-removal of skin tags. The kit contains sterilized forceps and scissors for grasping and severing a skin tag smaller than one centimeter in diameter. The kit also contains medications for the numbing of the skin tag before severing, for stopping any minor bleeding that might occur, and for bandaging the wound site. The system involves the numbing of the skin tag and surrounding tissue, then grasping the skin tag with the forceps to assert tension into the base of the skin tag, and then severing the base of the skin tag with the scissors. Aluminum chloride can be used to stop minor bleeding, and petrolatum or petroleum jelly covered by a sterile bandage can be used to minimize infection.

### CROSS-REFERENCE TO RELATED APPLICATIONS

This application claims domestic priority on U.S. Provisional Patent Application Ser. No. 63/163,625, filed on Mar. 19, 2021, the content of which is incorporated herein by reference.

### FIELD OF THE INVENTION

The invention disclosed in this application is directed generally to the removal of skin tags from the human body, and more particularly to a kit containing the components needed for a self-removal of skin tags, and a system for affecting the removal of skin tags.

### BACKGROUND OF THE INVENTION

Acrochordons, more commonly referred to as “skin tags”, are benign (non-cancerous) growths on the skin of humans that are connected to the skin by a small, thin stalk called a peduncle. Most skin tags are tiny, typically between two and five millimeters in size; however, some skin tags can grow as large a several centimeters. Skin tags are typically round, mobile, and soft to the touch. Skin tags may be smooth or wrinkly. Approximately sixty (60%) percent of the human population has skin tags. Skin tags are common in both men and women and are more prevalent in individuals over the age of fifty (50) years and in patients with diabetes.

Most skin tags are found in the armpits (axillae), groin, neck, eyelids or under the breasts. Skin tags often become irritated, inflamed and/or painful from rubbing against clothing, jewelry or skin folds. The removal of skin tags is not typically covered by insurance policies and can cost hundreds of dollars for removal by a board-certified dermatologist.

In U.S. Pat. No. 8,128,637, granted to Staffan Sundstrom on Mar. 6, 2012, a device for removing skin tags is disclosed as a device that is secured to the patient's skin and used to strangulate the skin tag by occluding the flow of blood thereto. The Sundstrom device covers and conceals the skin tag while being removed. In U.S. Pat. No. 10,542,997, granted to the University of Florida Research Foundation, Inc. on Jan. 28, 2020, a hemostatic clamp is provided to engage unwanted tissue, such as a skin tag, and maintain pressure until the skin is fused to create a skin tab which can then be excised with the clamp in position. The fusion of the skin limits bleeding upon excision. In U.S. Pat. No. 10,736,639, issued on Aug. 11, 2020, to Tagaway Devices, APS, an applicator is disclosed that includes a skin tag removal device that occludes the skin tag and interrupts the flow of blood thereto and allow the skin tag to be removed.

Other skin tag removal devices and processes are available for purchase over the internet. These devices include processes for cryogenic (freezing) removal and chemical removal such as with salicylic acid (frequently marketed for wart removal). A number of commercially available devices also use rubber bands to strangulate the skin tag. Still others utilize preparations that can include propylene glycol, tea tree oil, titanium dioxide, some of which are infused in patches applied to the skin tag and surrounding skin. These devices and preparations generally do not work, but cause swelling, irritation of the skin, and scabbing. At least one is not recommended to be suitable for patients with diabetes.

Accordingly, it would be desirable to provide a system and a kit for the self-removal of skin tags from easily reached locations on the body. Removal of skin tags from sensitive locations, particularly from around the eyes or the genitals, should be accomplished by a healthcare professional, particularly a board-certified dermatologist. This skin tag removal kit should also not be used to remove skin tags in children.

### SUMMARY OF THE INVENTION

It is an object of this invention to overcome the disadvantages of the known prior art by providing a convenient to use system and kit for the removal of skin tags.

It is another object of this invention to provide a system by which a person can effectively self-remove skin tags in a safe manner.

It is a feature of this invention that the kit disclosed herein provides the utensils and medication to permit a safe self-removal of skin tags.

It is an advantage of this invention that skin tags can be removed without incurring charges for professional help.

It is another feature that the disclosed kit for removing skin tags includes forceps and scissors for the mechanical removal of skin tags from the human body.

It is still another feature of this invention that the disclosed kit for removing skin tags includes medicinal ointments or liquids for the anesthetizing the skin tag, for stopping bleeding and for bandaging the removal site.

It is another advantage of this invention that once the forceps and scissors have been utilized in the removal of one or more skin tags, these instruments can be sterilized, stored and reused at a later date for removal of skin tags on the same individual.

It is yet another feature of this invention that the skin tag removal kit should not be utilized at home to remove skin tags having a diameter greater than one centimeter, as such large skin tags typically have a blood supply. Such large skin tags should be removed by a professional.

These and other objects, features and advantages are accomplished according to the instant invention by providing a system for a safe and effective removal of skin tags at home. A kit is also provided that contains the necessary instruments and medications for the safe and effective self-removal of skin tags. The kit contains sterilized forceps and scissors for grasping and severing a skin tag smaller than one centimeter in diameter. The kit also contains medications for the numbing of the skin tag before severing, for stopping any minor bleeding that might occur, and for bandaging the wound site. The system involves the numbing of the skin tag and surrounding tissue, then grasping the skin tag with the forceps to apply tension into the base of the skin tag, and then severing the base of the skin tag with the scissors. Aluminum chloride can be used to stop minor bleeding, and petrolatum or petroleum jelly covered by a sterile bandage can be used to prevent infection and promote healing of the skin.

### BRIEF DESCRIPTION OF THE DRAWINGS

The advantages of this invention will become apparent upon consideration of the following detailed disclosure of the invention, especially when taken in conjunction with the accompanying drawings wherein:

FIG. 1 is a perspective view of a kit for use for the self-removal of a skin tag, the contents of the case of the kit being shown in phantom lines;

FIG. 2 is an enlarged detail view of the respective medical contents that are found in the case; and

FIG. 3 is a logic flow diagram depicting the process for self-removal of skin tags utilizing the kit to create a skin tag self-removal system.

### DETAILED DESCRIPTION OF THE PREFERRED EMBODIMENT

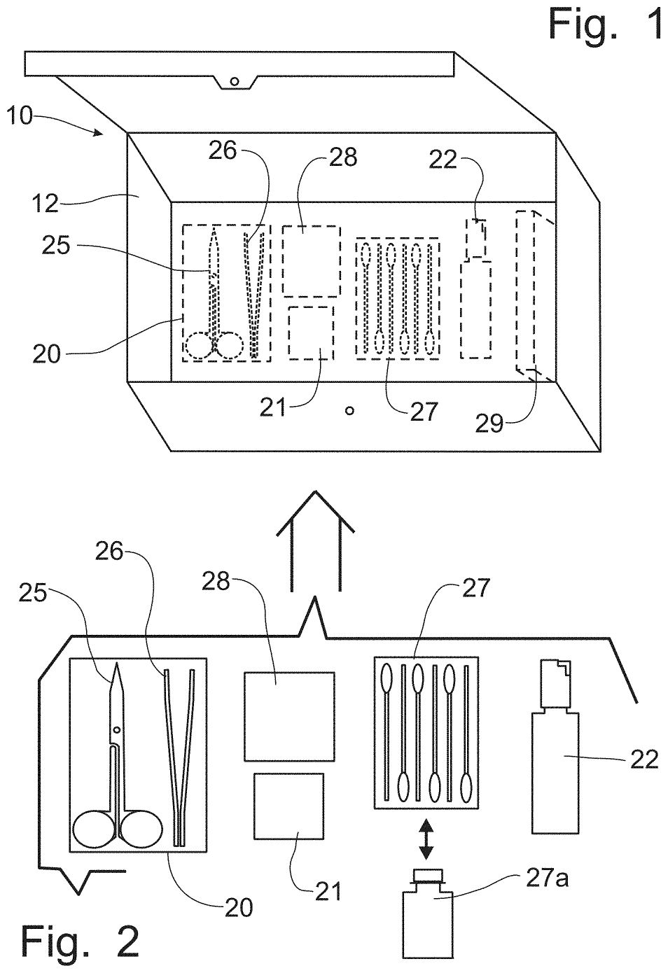

Referring to FIGS. 1-2, a kit for removing skin tags incorporating the principles of the instant invention can best be seen. The skin tag removal kit 10 includes a case 12 into which the components thereof can be stored. This skin tag removal kit 10 is intended for a person to self-remove skin tags using the tenets of the instant invention, although depending on the location of the skin tag to be removed, the user may require assistance in effecting the removal.

The skin tag removal kit 10 has some form of container 12, which is depicted in FIG. 1 as being a box with a lid, although one of ordinary skill in the art will recognize that the container 12 can be of substantially any form from an envelope or bag to a metal or plastic box. The intent of the container 12 is to store the components of the kit 10 for deployment when desired. Some of the components are preferably stored in sterile packages and when opened and deployed in the skin tag removal system that sterile condition is depleted. Accordingly, this skin tag removal kit 10 is primarily intended for a deployment one time. Multiple skin tags can be removed during that single deployment, and replacement sterile components can be acquired to permit a subsequent utilization of the skin tag removal kit 10.

This skin tag removal kit 10 can remove many, even hundreds, of skin tags during the single deployment, but the kit should not be shared by multiple people as to do so involves a risk of contamination and contraction of blood-borne diseases. The self-removal of a skin tag can be accomplished within a few minutes through use of this skin tag removal kit 10 in the comfort of the patient's own home. As a matter of warning, this skin tag removal kit 10 was not designed to be, or intended to be, used for the removal of moles, warts or any other growth on the patient's skin. Furthermore, this skin tag removal kit should not be used to remove skin tags that have a diameter of one centimeter (1 cm) or more, as the risk of bleeding is greater with such large skin tags.

As is depicted in FIGS. 1 and 2, the skin tag removal kit 10 preferably includes as components: alcohol preparation wipes 21; topical lidocaine preparation 22, or some form numbing solution; scissors 25; forceps 26; aluminum chloride solution with cotton swabs 27, or some form of solution or powder that will stop bleeding; gauze 28; written instructions and instructional video 29. Preferably, the scissors 25 and forceps 26 come with gauze 28 in a sterilized, sealed package 20, commonly referred commercially as a suture removal kit. The alcohol preparation wipes are provided commercially as individually packaged alcohol wipes, such as is available as Coviden Webcol wipes.

The aluminum chloride solution with cotton swabs 27 are available in a clean, pre-packaged array in which the cotton swabs are already impregnated with the aluminum chloride solution. Alternatively, a bottle of 20% aluminum chloride 27a can be provided within the kit 10 for administering to cotton-tipped swabs provide by the patient. There are other wound sealant powders and solutions that can be used to stop bleeding instead of the preferred aluminum chloride swabs. The numbing solution 22 is preferably in the form of a lidocaine numbing spray that can be applied to the skin tag and surrounding area, but other numbing solutions, such as Aspercreme® with lidocaine pain relieving crème. The skin tag removal kit 10 can also include bandages, and petrolatum for application to the wound site. However, such components are usually within the patient's normal medical supplies and would not be a required component for the skin tag removal kit 10.

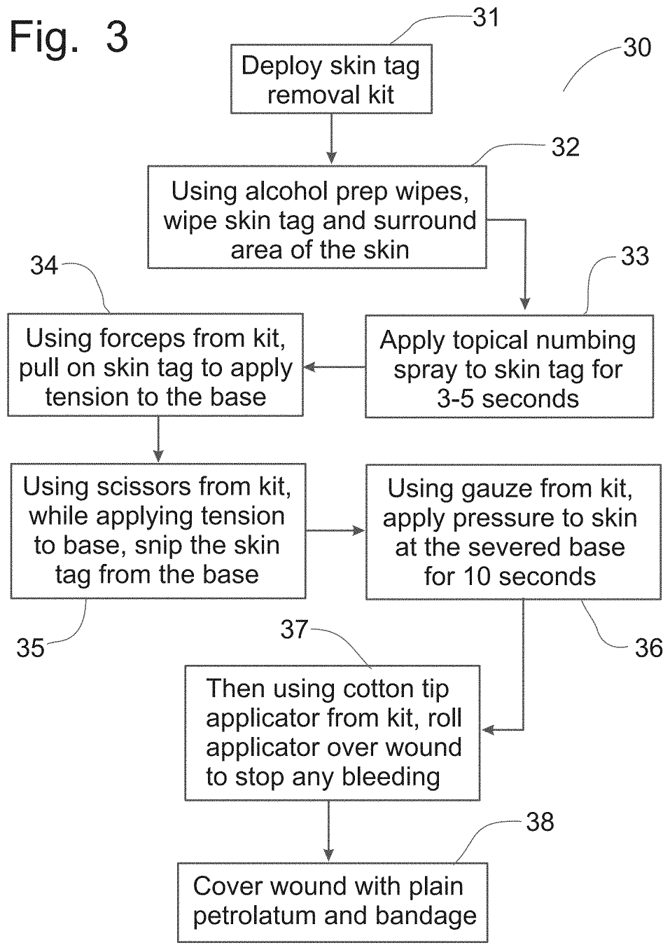

The system 30 for the self-removal of skin tags is documented in FIG. 3. Once the skin tag removal kit 10 is obtained and deployed at step 31, the patient is to open one or more alcohol preparation wipe packages at step 32 and remove the alcohol wipe to wipe and sterilize the skin tag to be removed and the surrounding skin. The sterile package 20 containing the scissors 25 and forceps 26 is then opened and removed from the package 20. Preferably, the patient uses the numbing spray (or other numbing solution) at step 33 to anesthetize the skin tag. The patient then utilizes the forceps at step 34 to grasp the skin tag to be removed and pull on it in a direction away from the patient's body in order to place some tension on the smaller base of the skin tag. While tension is being applied to the base of the skin tag, the scissors 25 are manipulated at step 35 to snip the skin tag from the body at the base. At this stage, the patient may need some additional help depending on the location of the skin tag as a simultaneous manipulation of both the forceps 26 and the scissors 25 to remove the skin tag may be difficult for the patient to accomplish alone.

Once the skin tag has been removed, the severed base may bleed, although small skin tags may not bleed at all. However, if some bleeding occurs, the patient should use the gauze from the skin tag removal kit 10 at step 36 to apply pressure on the wound for approximately ten (10) seconds, and then the solution to stop the bleeding should be applied at step 37 by either removing one of the clean pre-packaged swabs soaked with aluminum chloride, or opening a bottle of aluminum chloride and applying the liquid to a fresh cotton-tipped swab, and then roll the cotton-tipped swab over the wound to apply the aluminum chloride to the wound site and stop the bleeding. The wound can then be bandaged at step 38 after applying a dab of petroleum jelly to keep the wound moist.

As with all surgical procedures, whether minor surgery or major surgery, some risks are encountered. The following risks certainly apply to the utilization of the skin tag kit 10 for the self-removal of skin tags, including infection, bleeding, scar formation and allergies to the anesthetic. While infections are unlikely to occur from such a small wound as is encountered with the snipping of the base of a skin tag, cleaning the area with the alcohol preparation wipes and keeping the surrounding area clean after the skin tag is removed will decrease the risk of infection. Wound care should be performed for approximately 3-5 days following removal of the skin tag or until all scabbing resolves. Signs of infection would include, but not necessarily be limited to, redness of the skin surrounding the wound site, pain, drainage and fever.

The skin tag removal kit 10 includes devices and methods to stop bleeding from the wound site. If bleeding is unable to be stopped with the components 27, 27a included in the skin tag removal kit 10, the patient should hold pressure for twenty (20) minutes without relieving pressure. The patient may also apply ice to the area surrounding the wound at the same time as applying pressure. If bleeding still does not cease after these measures, then the patient should report to the closest emergency room. Large skin tags, i.e., skin tags greater than one centimeter (1 cm) should not be removed at home, as these large skin tags have a significant blood supply and should be treated by a medical professional in an office setting.

The above-described system 30 for the self-removal of skin tags is safe and is as effective as the removal of the skin tag at the office of a medical professional. Each skin tag can be removed in a few moments of time at a minimum of cost to the patient. Once the skin tag removal kit 10 has been deployed, subsequent use thereof should involve a re-sterilization of the scissors 25 and the forceps 26, which can be accomplished by wiping these metallic components with alcohol. The skin tag removal kit 10 should not be shared among individuals even after cleansing components with alcohol to avoid risk of contraction of blood borne illness.

It will be understood that changes in the details, materials, steps and arrangements of parts, which have been described and illustrated to explain the nature of the invention will occur to and may be made by those skilled in the art upon a reading of this disclosure within the principles of the scope of the invention. The foregoing description illustrates the preferred embodiment of the invention; however, concepts, as based upon the description may be employed in other embodiments without departing from the scope of the invention. For example, the vibration generator could be powered electrically instead of pneumatically. Accordingly, the following claims are intended to protect the invention broadly, as well as in the specific form shown.

Having thus described the invention, what is claimed is:

1. A skin tag removal kit comprising:
a container for storing individual components of said kit;
a pair of scissors;
a pair of forceps;
alcohol preparation wipes;
anesthetic numbing solution;
gauze; and
a solution to stop bleeding of a wound site after removal of the skin tag.

2. The skin tag removal kit of claim 1 wherein said scissors and said forceps are pre-packaged together in a sterile environment.

3. The skin tag removal kit of claim 2 wherein said scissors and said forceps are part of a sterilized suture removal kit.

4. The skin tag removal kit of claim 1 wherein said anesthetic numbing solution is a lidocaine numbing spray.

5. The skin tag removal kit of claim 1 wherein said solution to stop bleeding includes at least one cotton-tipped swab soaked with aluminum chloride.

6. The skin tag removal kit of claim 5 wherein said soaked cotton-tipped swabs are pre-packaged in a clean container.

7. The skin tag removal kit of claim 1 wherein bandages are also included.

8. The skin tag removal kit of claim 7 wherein petrolatum is included for application to the wound site before a bandage is applied.

9. A system for the self-removal of a skin tag having a base connecting to a patient's skin, comprising:
deploying a skin tag removal kit having a pair of scissors; a pair of forceps; alcohol preparation wipes; anesthetic topical numbing solution; gauze; and a solution to stop bleeding of a wound site after removal of the skin tag;
wiping the skin tag and the skin surrounding the skin tag with one of said alcohol preparation wipes;
applying said anesthetic topical numbing solution to the skin tag;
using the forceps, engaging the skin tag and pulling the skin tag away from the body to assert tension on the base of the skin tag;
while tension is asserted on the base of the skin tag, manipulating the scissors to snip the base of the skin tag away from the surrounding skin top create a wound site;
stopping any bleeding from the wound site with said solution to stop bleeding; and
bandaging said wound site.

10. The system of claim 9 wherein applying step includes the spraying of a lidocaine numbing spray on said skin tag and the surrounding skin.

11. The system of claim 9 wherein said stopping step includes the application of aluminum chloride to the wound site by utilizing a cotton-tipped swab soaked with the aluminum chloride.

12. The system of claim 9 wherein said scissors and said forceps are inside a sterile, pre-packaged suture removal kit prior to being deployed.

13. The system of claim 9 wherein said bandaging step includes an application of petrolatum or petroleum jelly on said would site before applying a bandage to said wound site.
